(19) United States
(12) Patent Application Publication
(10) Pub. No.: US 2009/0023692 A1
(43) Pub. Date: Jan. 22, 2009

(54) COMPOSITIONS FOR INHIBITING AND PREVENTING VASCULAR OCCLUSION CONTAINING NUTRITIONAL SUPPLEMENTS

(75) Inventor: Krishna Vishnupad, Dayton, OH (US)

Correspondence Address: SEYFARTH SHAW LLP, WORLD TRADE CENTER EAST, TWO SEAPORT LANE, SUITE 300, BOSTON, MA, 02210-2028, US

(73) Assignee: NeursoSci, Inc., Dayton, OH (US)

(21) Appl. No.: 11/816,987

(22) PCT Filed: Feb. 24, 2006
(86) PCT No.: PCT/US06/06733

§ 371 Date: Apr. 10, 2008

Related U.S. Application Data
(60) Provisional application No. 60/659,966, filed on Mar. 8, 2005.

(51) Int. Cl. A61K 31/60; A61P 9/00

(52) U.S. Cl. 514/165

(57) ABSTRACT

Compositions for inhibiting vascular occlusion in humans comprising aspirin for inhibiting vascular occlusion which further contain nutritional supplements are disclosed. The compositions are provide in a dosage form that provides stability of the active ingredient allowing for improved shelf life. A further composition included a cholesterol-lowering agent in combination with aspirin and optionally with nutritional supplements.

### FIELD OF THE INVENTION

The instant invention relates generally to compositions for preventing or inhibiting vascular occlusion in humans. In particular, it relates to compositions for preventing or inhibiting vascular occlusion containing nutritional supplements and various pharmaceutical active ingredients.

### BACKGROUND OF THE INVENTION

Atherosclerotic cardiovascular disease is a leading cause of death despite numerous therapeutics classes of prescription medications such as beta blockers, angiotensin converting enzyme inhibitors, cholesterol lowering medications and calcium channel blockers. Typically cardiovascular disease involves plaque formation in arteries through the body causing vascular occlusion. It has been shown that family history predisposes a person to cardiovascular disease, however, other risk factors include smoking, cholesterol abnormalities resulting from diet, environmental pollutants, hypertension and diabetes mellitus have emerged as leading risk factors.

Unfortunately, atherosclerotic cardiovascular disease greatly increase the risks of peripheral vascular disease, angina, stroke and heart attacks. In recent studies it has been shown that aspirin has a clear benefit in reducing the risk of stroke in younger women and an additional benefit of reducing the risk of heart attacks in older women.

Compositions for inhibiting platelet aggregation are known in the art. U.S. Pat. No. 5,401,730 to Sauvage, et al., ('730 patent) discloses a method of treating a patient comprising the administration of a combination of acetylsalicylic acid (aspirin), citric acid and thiamine, optionally in further combination with zinc. Unfortunately, the formulation as disclosed in the '730 patent is thought to be unstable as the combination of aspirin with the nutritional supplements has been shown to have a relatively short period of stability. Additionally, the plain uncoated aspirin within this composition may be harmful to the patient's gastrointestinal tract. Further the composition does not include some nutritional supplements that are thought to be beneficial to cardiac health.

Another prior attempt to incorporate aspirin with nutritional supplements is disclosed in U.S. Pat. No. 4,491,574 to Seifter, ('574 patent). The '574 patent discloses a therapeutic composition comprising aspirin in combination with vitamin A or a precursor of vitamin A to reduce toxicity and ulcerogenesis. As in the '730 patent, the stability of the composition of the '574 patent is relatively short and the plain uncoated aspirin potentially harmful to the patient's gastrointestinal tract. Further the composition, as in the '730 patent, does not include some nutritional supplements that are thought to be beneficial to cardiac health.

A further prior attempt to incorporate aspirin with nutritional supplements is disclosed in U.S. Pat. No. 6,274,170 ('170 patent), which discloses a therapeutic composition comprising aspirin in combination with various nutritional supplements. The '170 patent discloses a formulation having a protective coating around the aspirin and is thought to improve shelf life of the combination product, however, the formulation of the '170 patent is difficult and costly to manufacture on a commercial scale and the bio-availability of the aspirin component is suspect. Additionally, the composition does not include some nutritional supplements that are thought to be beneficial to cardiac health.

### SUMMARY OF THE INVENTION

The present invention is directed to compositions for preventing or inhibiting vascular occlusion in humans and more specifically to compositions for preventing or inhibiting vascular occlusion which further contain beneficial nutritional supplements.

An object of the present invention is to provide a single dosage formulation that contains both nutritional supplements and a vascular occlusion inhibitor in a stable form. The single dosage formulation increases patient convenience and compliance.

Another object of the present invention is to provide nutritional supplements in combination with aspirin (acetylsalicylic acid) and more specifically to combinations having enteric coated aspirin in a stable dosage form.

An additional object of the present invention is to provide vitamin supplements in combination with aspirin (acetylsalicylic acid) and nutritional minerals in a stable dosage form that prevents gastric upset.

Another object of the present invention is to provide vitamin supplements/vascular occlusion inhibitor which simplifies the process of treating cardiac care patients and improves patient compliance by combining aspirin and beneficial nutritional supplements into a single formulation.

A further object of the present invention is to provide a method of treating a cardiac care patient by administering to the patient vitamin supplements containing a vascular occlusion inhibitor such as aspirin in combination with cholesterol lowering agents.

A further object of the present invention is to provide a vitamin/mineral supplement in combination with aspirin for the combined effect of reducing inflammation and/or pain while providing nutritional supplementation.

An additional object of the present invention is to provide a vitamin/mineral supplement in combination with aspirin, with the aspirin useful for reducing heart attack damage, improving heart attack survival, reducing the incidence of second heart attacks and/or reducing death secondary to heart attacks.

Another object of the present invention is to provide a vitamin/mineral supplement in combination with aspirin and omega-3 fatty acids, with the aspirin useful for reducing or preventing strokes and/or transient ischemic attacks.

A further object of the present invention is to provide a vitamin/mineral supplement in combination with aspirin, cholesterol lowering agents and omega-3 fatty acids, with the aspirin useful for reducing or preventing strokes and/or transient ischemic attacks.

The foregoing and other objects, advantages and characterizing features will become apparent from the following description of certain illustrative embodiments of the invention.

The novel features which are considered characteristic for the invention are set forth in the appended claims. The invention itself, however, both as to its construction and its method use, together with additional objects and advantages thereof, will be best understood from the following description of the specific embodiments when read and understood in connection with the accompanying drawings. Attention is called to the fact, however, that the drawings are illustrative only, and that changes may be made in the specific construction illustrated and described within the scope of the appended claims.

### BRIEF DESCRIPTION OF DRAWINGS

The foregoing and other features and advantages of the present invention will be better understood from the following detailed description of illustrative embodiments, taken in conjunction with the accompanying drawings in which:

FIG. 1: depicts the co-packaging within a blister pack of an aspirin dosage form with a nutritional supplement dosage form;

FIG. 2: depicts a co-packaging within a two chamber container wherein one container contains an aspirin dosage form and the second container contains a nutritional supplement dosage form;

FIG. 3: depicts a tablet within a tablet formulation that contains an aspirin dosage layer and a second layer containing a nutritional supplement; and

FIG. 4: depicts a tablet within a capsule formulation that contains an aspirin dosage within mini-tablets surrounded by nutritional supplements within a gelatin capsule.

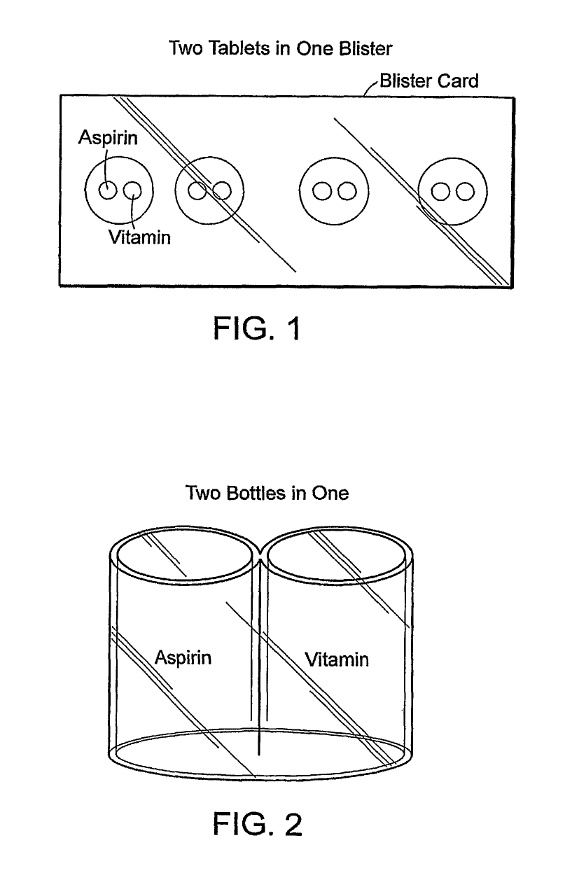

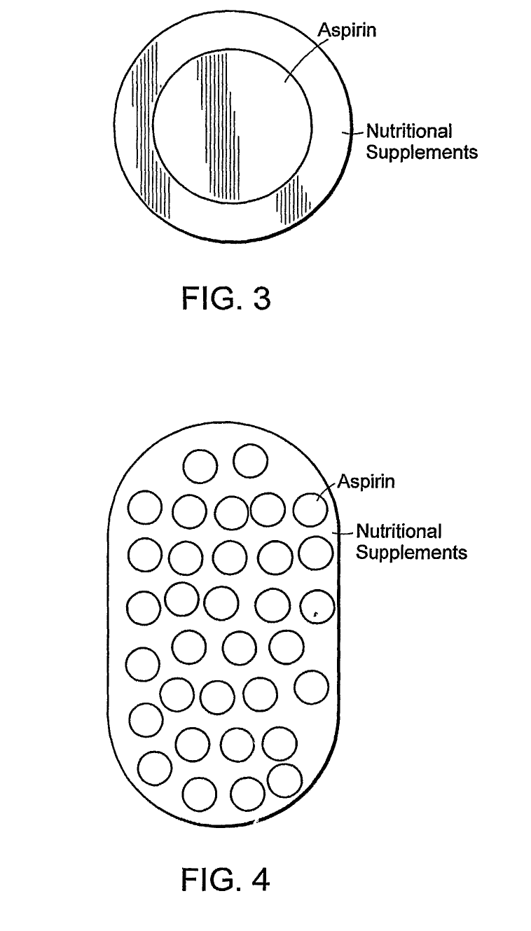

### DETAILED DESCRIPTION OF THE INVENTION

Detailed embodiments of the present invention are disclosed herein, however, it is to be understood that the disclosed embodiments are merely exemplary of the invention, which may be embodied in various forms. Therefore, specific functional details disclosed herein are not to be interpreted as limiting, but merely as a basis for the claims and as a representative basis for teaching one skilled in the art to variously employ the present invention in virtually any appropriately detailed embodiment.

Stability within drug manufacturing is a problem, which not only affects the shelf life of the finished drug blend or composition but also affects the effectiveness of the active ingredient within a final dosage form. When a drug, such as aspirin is placed in combination within other compounds such as nutritional supplements, it has been observed that the stability of the these formulations are short and are suspect. Special precautions have to be taken to maintain the stability of such combination formulations in order to provide a commercially viable shelf life.

According to a first illustrative embodiment of the present invention, a formulation includes a therapeutic effective amount of aspirin (acetylsalicylic acid) as an anti-platelet aggregating agent. The formulation in one illustrative embodiment comprises from about 25 to about 500 milligrams of aspirin and more preferably from about 50 to about 300 milligrams. In a first illustrative embodiment, the aspirin comprises about 100 milligrams of the dosage.

Aspirin when administered in low daily doses over a long term to patients at risk for cardiovascular events, is well established to prevent myocardial infarction and strokes due to thrombosis. It has been recently reported that second heart attacks, strokes, and cardiovascular deaths are reduced by about at least 25% through the daily administration of low doses (approximately 80-100 mg) of aspirin.

Without being bound to any particular theory it is thought that a number of mechanisms are likely responsible for the cardiovascular protective activity of aspirin, but its antithrombotic, anti-platelet aggregating activities are highly significant. Aspirin irreversibly acetylates the enzyme cyclooxygenase, rendering it nonfunctional. Cyclooxygenase is essential to the synthesis of (among other compounds) prostaglandins, many of which are pro-inflammatory; thromboxane A₂, which is synthesized by platelets to promote platelet aggregation and ultimately thrombosis (blood clotting); and prostacyclins, which have anti-platelet aggregating properties.

Low doses of aspirin neutralize cyclooxygenase selectively in the platelets, while allowing continued cyclooxygenase and prostacyclin synthesis in the endothelial cells. It is thought that this lead to reduced inflammation and platelet aggregation, and thus thrombosis, in blood vessels.

While in a first illustrative embodiment aspirin is used as an anti-platelet aggregating agents, it is contemplated within the scope of the invention that other salicylates, including magnesium salicylate may be used. It is further contemplated that other anti-platelet aggregating agents, such as anagrelide, dipyridamole, clopidogrel, and ticlopidine, may also be used. Other cyclooxygenase inhibitors, including other nonsteroidal anti-inflammatory drugs (NSAIDS) such as ibuprofen, sulindac, sulindac sulfide, sulindac sulfone, flurbiprofen, indomethacin, naproxen, meclafenamic acid, and piroxicam, are contemplated within the scope of the invention.

In accordance with a first illustrative embodiment of the present invention, the formulation is provided in a stable dosage form in which destructive interaction between the aspirin and the other active ingredients is substantially avoided. The stabilization is provided by having a protective coating between the aspirin and the additional nutritional supplements such as multiple vitamins.

In one illustrative embodiment, a protective enteric coating may be provided around the aspirin which minimizes or prevents deleterious reactions with the other vitamin and mineral ingredients. It is contemplated within the scope of the invention that the protective enteric coating can surround a tablet dose form of aspirin that is placed within a capsule or tablet or the protective coating can surround granulated aspirin that is placed within a capsule or tablet dosage form. It is further contemplated within the scope of the invention that an enteric coating may surround the aspirin component in a multi-layered tablet composition having a core formulation of an enteric coated aspirin surrounded by at least one additional layer having nutritional supplements. It is also contemplated within the scope of the invention that one layer or all layers within this construction may be instant release or controlled release. In a further illustrative embodiment enteric coated aspirin granules and enteric coated nutritional supplement granules are combined within a soft or hard gelatin capsule.

The protective coating preferably comprises at least one layer of wax, shellac, hydroxypropyl, methylcellulose phthalate, polyvinyl acetate phthalate, cellulose acetate phthalate and mixtures thereof. It is contemplated within the scope of the invention that other polymers known in the art may be used to coat the aspirin component in the formulation according to the invention.

The composition of the invention may optionally include pharmaceutically acceptable excipients. As is known in the art, pharmaceutical excipients are routinely incorporated into solid dosage forms. This is done to ease the manufacturing process as well as to improve the performance of the dosage form. Common excipients include diluents, lubricants, granulating aids, colorants, flavorants, surfactants, pH adjusters, anti-adherents and glidants. Such excipients are routinely used in the dosage forms of this invention.

The present invention may additionally include one or more fillers or excipients in an amount within the range of from about 0 to about 90% by weight and preferably from about 1 to about 80% by weight such as lactose, sugar, corn starch, modified corn starch, mannitol, sorbitol, inorganic salts such as calcium carbonate and/or cellulose derivatives such as wood cellulose and microcrystalline cellulose.

In one illustrative embodiment in the form of a tablet may include one or more tableting lubricants in an amount within the range of from about 0.2 to about 8% and preferably from about 0.5 to about 2% by weight of the composition, such as magnesium stearate, stearic acid, palmitic acid, calcium stearate, talc, polyethylene glycol, colloidal silicon dioxide, sodium stearyl fumarate, carnauba wax and the like and mixtures thereof. Other conventional pharmaceutical ingredients, which may optionally be present, include preservatives, stabilizers, and FD &C colors etc.

In a first illustrative embodiment, the protective coating comprises an enteric coating which may be applied to tablet formulations, mini-tablets or to drug particles or granules used in the subsequent fabrication of capsules or tablets. The coatings are applied in single or multiple stages depending upon the desired effect. The coating systems can be either aqueous based or organic solvent based to resist breakdown in the low pH environment of the stomach.

In the first illustrative embodiment, the formulation according to the invention may optionally include from about 90 to about 1,000 milligrams of vitamin C (ascorbic acid), more preferably from about 100 to about 150 milligrams. A particularly preferred amount of ascorbic acid is about 125 milligrams. It is contemplated that that ascorbic acid may be utilized as coated granules as is the aspirin to avoid stomach upset.

The first illustrative embodiment according to the invention may further optionally include Folic acid from about 400 to about 1,000 micrograms of the dosage, more preferably from about 400 to about 800 micrograms. It is thought that Folic acid acts as a coenzyme in DNA and RNA synthesis and is vital for protein metabolism. Without being bound to any particular theory, it is thought that the action of folic acid is enhanced when vitamins B₁₂ and C are present therapeutic levels.

The first illustrative embodiment according to the invention may further optionally include from about 5 to about 500 micrograms of vitamin B₁₂ (cyanocobalamin), more preferably from about 6 to about 100 micrograms. Vitamin B₁₂ is essential in the formation of red blood cells and augments the utilization of iron within the body. Vitamin B₁₂ is enhanced in conjunction with therapeutic levels of folic acid and vitamin B6.

The first illustrative embodiment according to the invention may further optionally include Vitamin B6 (pyridoxine) from about 3 to about 25 milligrams of the formulation, more preferably from about 6 to about 20 milligrams. In the first illustrative embodiment Vitamin B6 is present in about 6 milligrams.

The first illustrative embodiment according to the invention may further optionally include a therapeutically effective amount of a cholesterol-lowering agent or combination of such cholesterol-lowering agents. It is contemplated within the scope of the invention that cholesterol-lowering agents such as HMG CoA reductase inhibitors, bile acid sequestrants, probucol, and fibric acid agents may be used. In one illustrative embodiment HMG CoA reductase inhibitors are contemplated. It is contemplated that atorvastatin, cerivistatin, fluindostatin, fluvastatin, lovastatin, mevastatin, pravastatin, simvastatin, velostatin or the like may be included within the formulation within the above nutritional supplements and aspirin. It is further contemplated within the scope of the invention that cholesterol-lowering agents and aspirin may comprise the formulation according to the invention.

The first illustrative embodiment may optionally include omega-3 fatty acids. According to the invention the use of omega-3 fatty acids is not restricted to any one particular type of omega-3 fatty acid, it is preferred that eicosapentanoic acid (EPA) or docosahexanoic acid (DHA) be used or mixtures thereof. Both EPA and DHA are found in a variety of fish oils and are commercially available in an essentially pure form. It is thought that fatty acid compositions containing a high concentration, of at least 80% by weight, of omega-3 fatty acids, salts or derivatives thereof, where EPA and DHA are present in relative amounts of 1:2 to 2:1, and constitute at least 75% of the total fatty acids, have a surprisingly advantageous effect on all the above mentioned cardiac risk factors, but especially a good effect on mild hypertension, hypertriglyceridemia and on the coagulation factor VII phospholipid complex activity. It is thought that the use of omega-3 fatty acids lowers serum LDL-cholesterol, increases serum HDL-cholesterol, lowers serum triglycerides, lowers systolic and diastolic blood pressure and the pulse rate and lowers the activity of the blood coagulation factor VII-phospholipid complex.

Although the detailed biological mechanisms for the effects of the compositions according to present invention are not explicitly known, there are indications of a surprising synergism between the action of EPA and of DHA. An especially preferred composition according to the present application comprises at least 90% by weight of long chain, polyunsaturated omega-3 fatty acids of which EPA and DHA constitute at least 85% by weight of the total fatty acids and are present in a ratio of EPA:DHA from 1:1 to 2:1 especially about 3:2. It is contemplated within the scope of the invention that omega-3 fatty acids in the amount of approximately between 1 and about 2 grams may be optionally included within the formulation.

It is understood that DHA and EPA, in any of the above described forms, can be used individually, or as a mixture of the two. The mixtures are prepared by combining the desired quantities of the purified constituents; alternatively the DHA and EPA mixtures or the mixtures of their esters or salts thereof are used, as obtained by extraction, purification and concentration processes of the two acids starting from their natural sources (mainly fish oils). In a first illustrative embodiment the omega-3 fatty acids are incorporated into the combinations illustrated above by mini-soft gels within a capsule formulation. It is contemplated within the scope of the invention that a larger soft gel can constitute one-half of a capsule formulation within the other desired ingredients being contained in the other half of the capsule. The first illustrative embodiment according to the invention may further optionally include Vitamin B1 (thiamin) as it is thought to help keep the heart functioning normally.

The first illustrative embodiment according to the invention may further optionally include Niacin. It is thought that Niacin reduces high blood pressure, and increases energy through proper utilization of food.

As is known in the art, the body secretes any excess B-complex vitamins and thus does not store the same in the body. B-complex vitamins, particularly those described above, must be replaced daily to maintain good health. Various illustrative embodiments according to the invention provide this replacement.

The first illustrative embodiment according to the invention may further optionally include Magnesium in therapeutic amounts. It is thought that Magnesium can promote a healthier cardiovascular system, keep teeth healthier, and help prevent calcium deposits, kidney stones and gallstones.

The first illustrative embodiment according to the invention may further optionally include Zinc. It is thought that Zinc is essential for protein synthesis, governs the contractibility of muscles, helps in the formation of insulin, and is important for food stability and in maintaining the body's acid-alkaline balance.

The first illustrative embodiment according to the invention may further optionally include calcium. It is thought that Calcium and magnesium work together for cardiovascular health. Calcium itself helps to maintain strong bones and healthy teeth. Calcium promotes a regular heart beat, alleviates insomnia, and helps metabolize the body's iron.

The particular combinations in the first illustrative embodiment of Vitamin B₁₂, Vitamin B₆ and folic acid are for the treatment of elevated serum levels of one or more the metabolites homocysteine (HC), cystathionine (CT), methylmalonic acid (MMA), or 2-methylcitric acid (2-MCA). Without being bound to any particular theory, it is thought that the administration of a therapeutic amount the above vitamin formulations in combination with aspirin according to the invention provides better long-term normalization of serum HC and other metabolites, and eliminates the difficulty in differentiating between deficiencies of two or three of the vitamins and the difficulty in diagnosing multiple deficiencies of two or three of the vitamins. It is contemplated within the scope of the invention that the administration of an oral preparation of B₁₂ and folic acid, with or without B₆, is preferred over intramuscular injections of B₁₂ for patient convenience and ease of administration.

The inclusion of B₁₂ is useful as a safeguard for patients misdiagnosed as folate deficient, even though they are actually B₁₂ deficient, since treatment with folic acid alone in such patients may be detrimental. The inclusion of folic acid within a first illustrative embodiment will be of benefit since B₁₂ deficiency causes a secondary intracellular deficiency of folate. The inclusion of folic acid and B₆ will also be of benefit in patients with mixed vitamin deficiencies.

The composition made according to the present invention may be formulated as tablets within a capsule, tablets within a tablet or multi-layered tablets. The tablet may optionally be coated with a thin layer of a film forming polymer or a pharmaceutical excipient. The controlled release preparation according to the invention may conveniently be film coated using any film coating material conventional in the pharmaceutical art. Preferably an aqueous film coating is used.

Oral dosage forms are used to administer the combination of active agents, and include tablets, capsules, caplets, solutions, suspensions, and/or syrups, and may also comprise a plurality of granules, beads, powders, mini-tablets, mini-soft gels or pellets that may or may not be encapsulated.

Tablets may be manufactured using standard tablet processing procedures and equipment. One method for forming tablets is by direct compression of a powdered, crystalline, or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

In addition to the active agent(s), tablets prepared for oral administration using the method of the invention will generally contain other materials such as binders, diluents, lubricants, disintegrants, fillers, stabilizers, surfactants, coloring agents, and the like. Binders are used to impart cohesive qualities to a tablet, and thus ensure that the dosage form remains intact after compression. Suitable binder materials include, but are not limited to, starch (including corn starch and pre-gelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum.

Diluents are typically necessary to increase bulk so that a practical desirable size tablet is ultimately provided. Suitable diluents include dicalcium phosphate, calcium sulfate, lactose, cellulose, kaolin, mannitol, sodium chloride, dry starch, and powdered sugar. Lubricants are used to facilitate tablet manufacture; examples of suitable lubricants include, for example, magnesium stearate, calcium stearate, and stearic acid. Stearates, if present, preferably represent at no more than approximately 2 wt. % of the drug-containing core. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers are used to inhibit or retard drug decomposition reactions that include, by way of example, oxidative reactions. Surfactants may be anionic, cationic, amphoteric, or nonionic surface active agents.

The dosage form may also be a capsule, in which case the active agent-containing composition may be encapsulated in the form of a liquid or solid (including particulates such as granules, beads, powders, or pellets). Suitable capsules may be either hard or soft, and are generally made of gelatin, starch, or a cellulosic material, with gelatin capsules preferred. It is contemplated within the scope of the invention that tablets or mini-tablets may be placed inside a capsule along with other active ingredients.

As is known in the art, when two or more active agents are combined in a single pharmaceutical dosage form, possible interactions among the active agents, and among the active agents and the excipients, must be considered. In the instant disclosure it should be noted that, aspirin is acidic and may react with basic compounds or alkali esters in such a way as to cause hydrolysis of the aspirin and/or degradation of the other compounds. The present composition thus encompasses pharmaceutical compositions wherein two or more of the active agents are separated from each other within the pharmaceutical dosage form, by, for example, separating potentially interacting compounds from each other within the pharmaceutical dosage form, as in separate flat layers of a tablet (e.g., a bilayer, trilayer multi-layer tablet), concentric layers, coated beads, min-tablets, min-soft gels or granules (which may be incorporated into a compressed tablet or into a capsule), and/or by using buffers. It will also be appreciated by those skilled in the art that such dosage forms, wherein two or more active agents are physically separated from the other active agents, can be manufactured so that different active agents will have different release profiles, e.g., if one active agent is formulated with an enteric coating, another active agent is formulated in a sustained release matrix, and the like. Alternatively, non-reactive pharmaceutically active derivatives of one or more of the potentially interacting compounds may be used, such as using a neutral salicylate instead of aspirin.

Solid dosage forms, whether tablets, capsules, caplets, or particulates, may, if desired, be coated so as to provide for delayed release. Dosage forms with delayed release coatings may be manufactured using standard coating procedures and equipment. Generally, after preparation of the solid dosage form, a delayed release coating composition is applied using a coating pan, an airless spray technique, fluidized bed coating equipment, or the like.

Delayed release coating compositions comprise a polymeric material including but not limited to cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof and mixtures thereof.

Sustained release dosage forms provide for drug release over an extended time period, and may or may not be delayed release. Typically, sustained release dosage forms are formulated by dispersing a drug within a matrix of a gradually bioerodible (hydrolyzable) material such as an insoluble plastic, a hydrophilic polymer, or a fatty compound, or by coating a solid, drug-containing dosage form with such a material. Insoluble plastic matrices may be comprised of, for example, polyvinyl chloride, polyethylene or the like.

Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexyhydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate, and trimethylammonioethyl methacrylate chloride (sold under the tradename Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate. IV.

It is preferred for patient compliance and convenience that the active agents be administered in a single dosage form, as emphasized and discussed above. However, in some cases, a patient may be given each active agent in its own separate dosage form, or a combination of individual “combination” dosage forms containing two or more of the present active agents. When separate dosage forms are used, the aspirin, and the nutritional supplements can be administered at essentially the same time, or at separately staggered times. It is contemplated within the scope of the invention that when separate dosage forms are utilized that the dosage forms will be blister packed and optionally colored coded or having different tablet shapes in a manner that improves patient compliance. In one illustrative embodiment the nutritional supplements are formulated in a separate tablet dosage form and the aspirin is accordingly formulated in an enteric coated dosage form. The two separate dosage forms are then packaged in a blister pack in a manner that improves patient compliance.

Preferred oral dosage forms contain a therapeutically effective unit dose of each active agent, wherein the unit dose is suitable for once-daily oral administration. Those of ordinary skill in the art of pharmaceutical formulation can readily deduce suitable unit doses for various active agents. In general, however, the therapeutically effective unit dosages for each of the active agents are as follows:

Approximately 20 mg to approximately 600 mg and preferably approximately 20 mg to approximately 150 mg, of aspirin.

Optionally, at least one of: approximately 5 mg to approximately 75 mg and preferably approximately 5 mg to approximately 40 mg, of vitamin B₆; approximately 0.5 mcg to approximately 20 mcg and preferably approximately 0.5 mcg to approximately 10 mcg, of vitamin B₁₂; and approximately 200 mcg to approximately 1000 mcg and preferably approximately 400 mcg to 800 mcg, of folic acid.

In a first illustrative embodiment, the active ingredients are as follows:

81 mg of aspirin;

6 mg of vitamin B₆;

6 mcg of vitamin B₁₂; and

400 mcg of folic acid.

In a second illustrative embodiment, the active ingredients are as follows:

Pravastatin 40 mg; and

81 mg aspirin.

In a third illustrative embodiment, the active ingredients are as follows:

Pravastatin 40 mg;

81 mg Aspirin;

Vitamin B₆ 6 mg;

Vitamin B₁₂ 6 mcg; and

400 mcg of folic acid.

In a fourth illustrative embodiment, the active ingredients are as follows:

100 mg of aspirin;

6 mg of vitamin B₆;

6 mcg of vitamin B₁₂; and

400 mcg of folic acid.

In a fifth illustrative embodiment, the active ingredients are as follows:

Pravastatin 40 mg; and

100 mg aspirin.

In a sixth illustrative embodiment, the active ingredients are as follows:

Pravastatin 40 mg;

100 mg Aspirin;

Vitamin B₆ 6 mg;

Vitamin B₁₂ 6 mcg; and

400 mcg of folic acid.

In a seventh illustrative embodiment, the active ingredients are as follows:

100 mg of aspirin;

6 mg of vitamin B₆;

6 mcg of vitamin B₁₂;

400 mcg of folic acid; and

a therapeutic amount of omega-3 fatty acids.

In a eight illustrative embodiment, the active ingredients are as follows:

Pravastatin 40 mg;

100 mg aspirin; and

a therapeutic amount of omega-3 fatty acids.

In a ninth illustrative embodiment, the active ingredients are as follows:

Pravastatin 40 mg;

100 mg Aspirin;

Vitamin B₆ 6 mg;

Vitamin B₁₂ 6 mcg;

400 mcg of folic acid; and

a therapeutic amount of omega-3 fatty acids.

It will be understood that various modifications may be made to the embodiments and examples disclosed herein. Therefore, the above description and examples should not be construed as limiting, but merely as exemplification of the various embodiments. Those skilled in the art will envision other modifications within the scope and spirit of the claims appended hereto.

What is claimed is:

1. A method for treating a patient having an elevated cardiovascular risk, comprising orally administering, a pharmaceutical composition comprising:
a therapeutically effective amount of aspirin;
at least one of vitamin B₆, vitamin B₁₂, and folic acid; and
a pharmaceutically acceptable carrier, wherein each of the therapeutically effective unit dosages is a therapeutically effective daily dose.

2. The method according to claim 1 wherein said pharmaceutical composition further comprises a therapeutic amount of omega-3 fatty acids.

3. The method according to claim 1 wherein said composition contains said aspirin is in the amount of about 81 mg to about 100 mg.

4. The method according to claim 1 wherein said composition further contains minerals.

5. The method according to claim 4 wherein said minerals are selected from the group consisting of zinc, magnesium, calcium and iron.

6. The method according to claim 1 wherein said composition further comprises a cholesterol lowering agent.

7. A composition for treating a patient having an elevated cardiovascular risk, comprising:
a therapeutically effective amount of aspirin;
at least one of vitamin B₆, vitamin B₁₂, and folic acid; and
a pharmaceutically acceptable carrier, wherein each of the therapeutically effective unit dosages is a therapeutically effective daily dose.

8. The composition according to claim 7 wherein said pharmaceutical composition further comprises a therapeutic amount of omega-3 fatty acids.

9. The composition according to claim 7 wherein said composition contains said aspirin is in the amount of about 81 mg to about 100 mg.

10. The composition according to claim 7 wherein said composition further contains minerals.

11. The composition according to claim 10 wherein said minerals are selected from the group consisting of zinc, magnesium, calcium and iron.

12. The composition according to claim 7 wherein said composition further comprises a cholesterol lowering agent.

13. A composition for treating a patient having an elevated cardiovascular risk, comprising:
a therapeutically effective amount of aspirin;
a therapeutically effective amount of a cholesterol-lowering agent; and
a pharmaceutically acceptable carrier, wherein each of the therapeutically effective unit dosages is a therapeutically effective daily dose.

14. A packaged drug delivery kit for preventing cardiovascular incidents said kit including a container housing a plurality of oral dosage forms and instructions for carrying out drug administration therewith comprising:
a therapeutically effective daily dose of aspirin; and a therapeutically effective daily dose of at least one of vitamin B₆, vitamin B₁₂, and folic acid.
